(19) United States
(12) Patent Application Publication
(10) Pub. No.: US 2014/0096626 A1
(43) Pub. Date: Apr. 10, 2014

(54) SYSTEM FOR MEASURING THE PRECIOUS METAL MASS IN AN ALLOY

(71) Applicant: JOHN RALPH BICH, SUGAR LAND, TX (US)

(72) Inventor: JOHN RALPH BICH, SUGAR LAND, TX (US)

(21) Appl. No.: 14/017,609

(22) Filed: Sep. 4, 2013

Related U.S. Application Data
(60) Provisional application No. 61/712,055, filed on Oct. 10, 2012.

(51) Int. Cl. G01N 33/20

(52) U.S. Cl. CPC G01N 33/20
USPC 73/865

(57) ABSTRACT

A system to determine the mass of a precious metal existing with an alloy specimen where the purity concentration of the precious metal within the alloy is known by a user is provided. The system includes an assembly for weighing the alloy specimen, the assembly including an adjustable setting to permit the user to input the known purity concentration, a processor programmed to calculate the mass of the precious metal based upon the weight of the alloy and the purity concentration, and a display for visually displaying the precious metal mass to the user.

### RELATED APPLICATION

The application claims priority to provisional patent application U.S. Ser. No. 61/712,055 filed on Oct. 10, 2012, the entire contents of which is herein incorporated by reference.

### BACKGROUND

The embodiments herein relate generally to systems for determining the purity of a precious metal in an alloy. More specifically, embodiments of the invention relate to a system for measuring the mass of gold in an alloy specimen.

The purity and amount of a precious metal such as gold in an alloy is of great interest to retailers, collectors, customers, or the like. The amount of precious metal in the alloy specimen determines the market value of the alloy specimen. Currently, there exist several systems for determining the purity of a precious metal in a specimen. Systems for determining the purity of gold in a specimen include acid solution tests, electronic tests that require the specimen to be submerged in a liquid, and x-ray fluorescence spectrometers. However, these current systems are limited because they may damage the specimen, are not user-friendly, and/or require the use of expensive equipment.

As such, there is a need in the industry for a cost-effective system for measuring the mass of gold in an alloy specimen that is effective, easy to use and does not require chemicals that may damage the specimen.

### SUMMARY

A system configured to determine the mass of a precious metal existing with an alloy specimen where the purity concentration of the precious metal within the alloy is known by a user is provided. The system comprises an assembly for weighing the alloy specimen, the assembly comprising an adjustable setting configured to permit the user to input the known purity concentration, a processor configured and programmed to calculate the mass of the precious metal based upon the weight of the alloy and the purity concentration and a display for visually displaying the precious metal mass to the user.

### BRIEF DESCRIPTION OF THE FIGURES

The detailed description of some embodiments of the invention will be made below with reference to the accompanying figures, wherein the figures disclose one or more embodiments of the present invention.

FIG. 1 depicts a schematic side view of certain embodiments of the invention; and

FIG. 2 depicts a schematic side view of certain alternative embodiments of the invention.

### DETAILED DESCRIPTION OF CERTAIN EMBODIMENTS

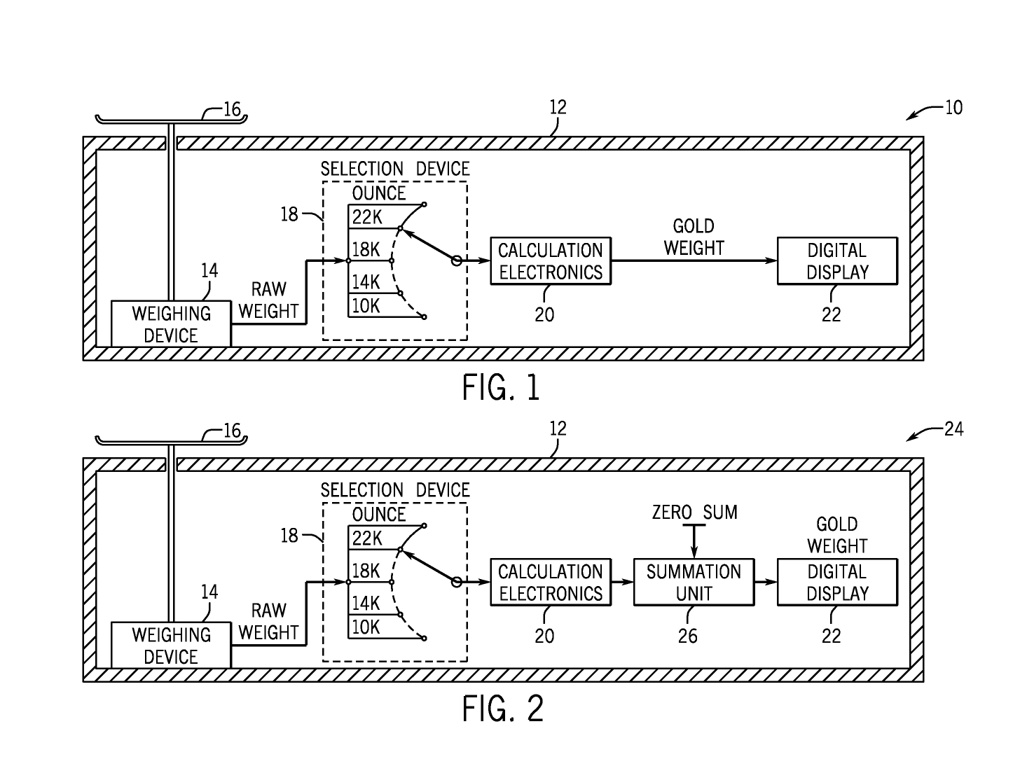

FIGS. 1 and 2 depict certain embodiments of the system for determining the mass of gold in an alloy specimen. The system 10 comprises housing 12, weighing device 14, tray 16, selection device 18, calculation electronics 20 and digital display 22. Weighing device 14 may be any type of scale known in the field that is configured to accurately measure the weight of a specimen. Selection device 18 may comprise any electronic or mechanical devices known in the field, which allow a user to select a known purity concentration of gold in the alloy specimen. The user inputs a karat value corresponding to the specimen using selection device 18, which may include 10K, 14K, 18K, 22K or 24K. Calculation electronics 20 is operably connected to weighing device 14 and selection device 18, and may include any processor and/or modules configured to calculate the mass or weight of gold in the alloy specimen. Digital display 22 may include any type of display known in the field including, but not limited to, LCD screens, LED screens, touch screens, or the like.

FIG. 2 depicts an alternative embodiment of the invention in which system 24 determines the total mass of gold in multiple alloy specimens. System 24 comprises summation unit 26, which may include any electronics and/or modules configured to sum the total mass of gold in multiple alloy specimens that are weighed using weighing device 14.

In certain embodiments of the invention, system 10 and system 24 may include a zero input selection button that allows a user to clear previous mass and/or weight values received by calculation electronics 20 related to a precious metal and alloy specimen.

In operation, a user places a gold alloy specimen on tray 16. Weighing device 14 determines the raw weight of the alloy specimen. The user selects the gold purity concentration in karats of the alloy specimen via selection device 18. The raw weight of the alloy specimen and the gold purity concentration number in karats is transmitted to calculation electronics 20. To determine the mass of gold in the alloy specimen, calculation electronics 20 multiplies the raw weight of the alloy specimen by a ratio comprising the karat value of the alloy specimen over 24. The mass of gold in the alloy specimen is transmitted to digital display 22, which is presented to the user.

The system is configured to calculate the total mass of gold in multiple alloy specimens. If the multiple alloy specimens each contain the same purity concentration of gold in karats, the user can place all of the specimens on tray 16 at once. In this setup, calculation electronics 20 determines the total mass of gold existing in the alloy specimens as described above. In the alternative, the user may place one alloy specimen on tray 16 at a time until all of the alloy specimens are weighed. This is necessary when at least two of the alloy specimens contain different karat values. Therefore, the user may separately weigh each alloy specimen and select the corresponding karat value for the specimen until all of the alloy specimens are weighed. It shall be appreciated that the user may weigh each alloy specimen separately in this manner even if all of the alloy specimens contain the same karat value. Calculation electronics 20 calculates the mass of gold in each alloy specimen and summation unit 26 sums all of the mass values to determine the total mass of gold existing in the alloy specimens. The total mass of gold is transmitted to digital display 22.

It shall be understood that the systems described herein may determine the mass of any other metal existing in an alloy specimen including, but not limited to, silver, platinum, palladium, copper, nickel, or the like.

It shall be appreciated that the components of the systems for measuring the mass of gold in an alloy specimen described in several embodiments herein may comprise any known materials in the field and be of any color, size and/or dimensions. In addition the components may be manufactured and/or assembled using any known techniques in the field.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A system configured to determine the mass of a precious metal existing with an alloy specimen where the purity concentration of the precious metal within the alloy is known by a user, the system comprising an assembly for weighing the alloy specimen, the assembly comprising:
an adjustable setting configured to permit the user to input the known purity concentration;
a processor configured and programmed to calculate the mass of the precious metal based upon the weight of the alloy and the purity concentration; and
a display for visually displaying the precious metal mass to the user.

2. The system of claim 1, wherein the assembly is configured to weigh multiple alloy specimens with each specimen having the same purity concentration of the precious metal and the processor comprises a summation unit configured to calculate the total mass of the precious metal in the multiple alloy specimens.

3. The system of claim 1, wherein the assembly is configured to weigh multiple alloy specimens where at least two alloy specimens contain different purity concentrations of the precious metal and the processor comprises a summation unit configured to calculate the total mass of the precious metal in the multiple alloy specimens.

4. The system of claim 1, wherein the assembly comprises a zero input selection button that is configured to clear previous mass and weight values received by the processor related to the precious metal and alloy specimen.

5. The system of claim 1, wherein the processor is configured to calculate the mass of the precious metal in the alloy specimen by multiplying the weight of the alloy specimen by a ratio based upon the known purity concentration.

6. The system of claim 5, wherein the alloy specimen comprises a portion of gold and the adjustable setting is configured to permit the user to input the known purity concentration of the gold in karats.

7. The system of claim 6, wherein the ratio comprises the fraction of karat value of the alloy specimen over 24.
